(19) United States
(12) Patent Application Publication
(10) Pub. No.: US 2025/0041560 A1
(43) Pub. Date: Feb. 6, 2025

(54) REMOVABLE LUNG ISOLATION DEVICE FOR LOCALIZED DRUG THERAPY

(71) Applicant: Covidien LP, Mansfield, MA (US)

(72) Inventor: Scott E.M. Frushour, Boulder, CO (US)

(73) Assignee: Covidien LP, Mansfield, MA (US)

(21) Appl. No.: 18/752,565

(22) Filed: Jun. 24, 2024

Related U.S. Application Data
(60) Provisional application No. 63/530,658, filed on Aug. 3, 2023.

(51) Int. Cl. A61M 25/00; A61M 25/10

(52) U.S. Cl. CPC A61M 25/0026; A61M 25/10; A61M 2025/105

(57) ABSTRACT

A system and method of applying therapy to a patient including navigating a dual lumen catheter to a desired segmental bronchus, inflating a balloon located on a distal portion of the catheter via a first tube of the dual lumen catheter to isolate the segmental bronchus, injecting a therapeutic medium into the isolated segmental bronchus via a second tube of the dual lumen catheter, and retracting the dual lumen catheter to leave the balloon indwelling, wherein the balloon retains the injected therapeutic medium in the isolated segmental bronchus.

### BACKGROUND

### Technical Field

This disclosure relates to the field of localized therapy, particularly localized therapy of lung tissues using chemical ablation techniques.

### Description of Related Art

The use of systemic therapies (e.g., chemotherapies) is well known and has proven beneficial for many patients in treating cancers within the body. Indeed, in many instances because of the nature of the tumor, its location, and neighboring tissues systemic therapies are currently the only mechanism of therapy. However systemic therapies are not without their downsides for the patient. The systemic treatment puts tremendous strain on the patient's immune system, often leaving them susceptible to other diseases, infections, and sepsis to say nothing of the pain and stress of these systemic therapies. In many instances the therapy leaves loved ones wondering whether the “cure” was worse than the disease.

Where possible, surgical and radiation approaches have long been identified as a logical first step in removing cancerous growths and tumors. However, that is not always possible. In other instances, such as in treating tumors of the lungs, the surgical approach (e.g., resection) to remove the diseased portion of the lungs can be quite challenging owing to the possibility of pneumothorax, the blood vessel rich tissues of the lungs themselves, and the difficulties in accurately assessing segmental portions of the lungs while performing the surgeries.

Further, even where surgical approaches like resection are employed, the systemic therapy is often employed after the fact as a prophylactic to ensure that all the cancerous cells have been eliminated. Thus, some of the benefits of the more targeted surgical approach are eliminated.

This disclosure is directed at addressing the shortcomings of both the systemic and the surgical approaches to cancer treatment, particularly for lung cancer.

### SUMMARY

One aspect of the disclosure is directed to a method of applying therapy. The method includes navigating a dual lumen catheter to a desired segmental bronchus. The method also includes inflating a balloon located on a distal portion of the catheter via a first tube of the dual lumen catheter to isolate the segmental bronchus; injecting a therapeutic medium into the isolated segmental bronchus via a second tube of the dual lumen catheter; and retracting the dual lumen catheter to leave the balloon indwelling, where the balloon retains the injected therapeutic medium in the isolated segmental bronchus. Other embodiments of this aspect include corresponding computer systems, apparatus, and computer programs recorded on one or more computer storage devices, each configured to perform the actions of the methods and systems described herein.

Implementations of this aspect of the disclosure may include one or more of the following features. The method where inflation of the balloon engages securing features with the segmental bronchus to hold the balloon in place. The securing features are micro-barbs. The securing features are an adhesive coating formed on an exterior surface of the balloon that adheres to an airway wall of the segmental bronchus. The method further including imaging the segmental bronchus with the balloon indwelling to assess the progression of the therapy. The method further including determining whether the therapeutic medium is draining from the segmental bronchus. The biological marker is configured to bind with a tumor or lesion. The method further including navigating the dual lumen catheter to the indwelling balloon. The first tube is configured to supply additional inflation medium into the balloon. The method further including passing the second tube through a second valve on the balloon. The method further including injecting additional therapeutic medium into the isolated segmental bronchus. The method further including extracting the therapeutic medium from the segmental bronchus. The method further including deflating the balloon. The deflation includes applying a vacuum to the balloon, where the vacuum secures the balloon to a portion of the dual lumen catheter. The method further including extracting the dual lumen catheter and balloon from the segmental bronchus. Implementations of the described techniques may include hardware, a method or process, or computer software on a computer-accessible medium, including software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

A further aspect of the disclosure is directed to a therapy application system having a dual lumen catheter including a first tube and a second tube, the dual lumen catheter configured for navigation within airways of a patient; a balloon formed on a distal portion of the dual lumen catheter, the balloon including a first valve on a proximal portion of the balloon and a second valve on a distal portion of the balloon; an inflation source in fluid communication with the first tube; and a therapeutic medium source in fluid communication with second tube, where the dual lumen catheter extends past the first valve and into the balloon, the first tube being in fluid communication with the balloon and release of inflation medium from the inflation source expands the balloon and isolates a segmental bronchus, and where the second tube extends beyond the second valve and release of the therapeutic medium from the therapeutic medium source floods the isolated segmental bronchus. Other embodiments of this aspect include corresponding computer systems, apparatus, and computer programs recorded on one or more computer storage devices, each configured to perform the actions of the methods and systems described herein.

Implementations of this aspect of the disclosure may include one or more of the following features. The therapy application system further including a vacuum source in fluid communication with the second tube and configured to extract the therapeutic medium from the isolated segmental bronchus. The vacuum source is in fluid communication with the first tube to deflate the balloon. The vacuum applied to the balloon secures the balloon to the dual lumen catheter for removal from the airways of the patient. The securing features is one or more of an adhesive, micro-barbs, hooks, or spikes. Implementations of the described techniques may include hardware, a method or process, or computer software on a computer-accessible medium, including software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

### BRIEF DESCRIPTION OF THE DRAWINGS

Various aspects and embodiments of the disclosure are described hereinbelow with references to the drawings, wherein:

FIG. 1 is a schematic view of a navigation system in accordance with the disclosure;

FIG. 2 depicts a catheter in accordance with the disclosure navigated to an individual segment of the left upper lobe of the lungs of a patient;

FIG. 3A is a schematic view of a catheter and an indwelling occluding device placed in an airway for application of localized therapy in accordance with the disclosure;

FIG. 3B is the schematic view of the indwelling occluding device with the catheter removed;

FIG. 4 is a view of a valve in accordance with the disclosure;

FIG. 5 is a flow chart of a method in accordance with the disclosure; and

FIG. 6 is a schematic view of a computing system in accordance with the disclosure.

### DETAILED DESCRIPTION

The disclosure is directed to systems and methods of localized therapy, particularly localized lung therapy. In accordance with the disclosure, a catheter is navigated to a location within the lungs in which a tumor has been identified. Once located at, near, or just beyond an entrance to an individual segment of the lung, a segment in which a tumor has been identified, a position that may be confirmed with imaging, an indwelling occluding device on the catheter is expanded (e.g., inflated) and a drug, chemical ablation medium, chemotherapy, or other therapeutic medium is passed through the expanded indwelling occluding device where it acts on the tumor and other cancerous cells within that segment of the lungs. The catheter is removed, leaving the indwelling occluding device in place while the therapy is allowed to act. After a specified period of time (e.g., 2-4 weeks) an assessment can be made regarding the tumor (e.g., with imaging) to assess whether additional therapy should be applied to the segment through the indwelling occluding device as described above. If no additional therapy is needed, the catheter is reinserted into the lungs of the patient. Any remaining therapy medium may be suctioned from the segment to remove it and other cellular tissue from the patient. The indwelling occluding device is then deflated and removed from the lungs with the catheter.

By this method, the long-acting benefits of systemic therapies are employed in a confined location allowing them to act only on the tissues in actual need of their actions. In doing so, many of the negative effects of systemic treatments and the burden on the patient's immune system are greatly reduced. Further, the patient has a narrowed impact to their respiratory system with the occlusion of just the impacted segment(s) of the lungs, thus their quality of life during treatment is improved. Still further, the dangers associated with a surgical approach such as resection are eliminated entirely. And there is likely no need for any post-surgical prophylactic systemic treatment.

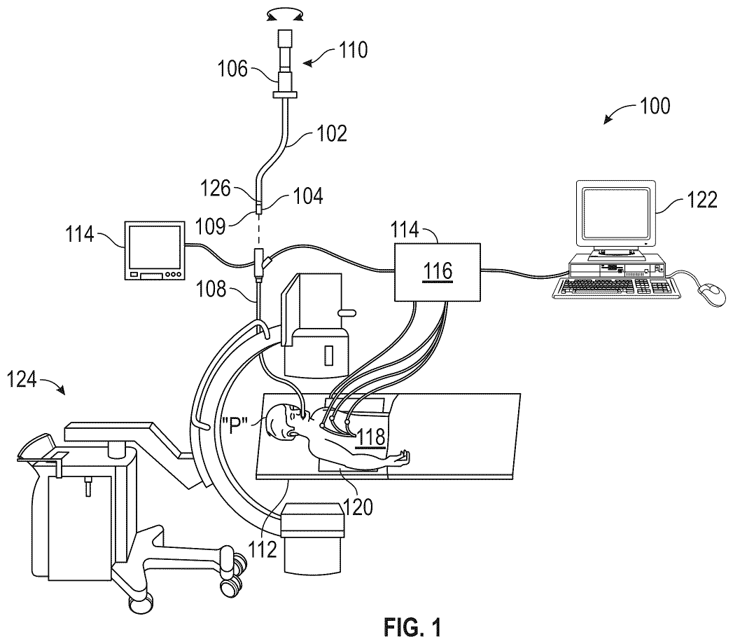

FIG. 1 is a perspective view of an exemplary system for facilitating navigation of a catheter to a soft tissue target via airways of the lungs. As shown in FIG. 1, catheter 102 is part of a catheter guide assembly 106. In one embodiment, catheter 102 is inserted into a bronchoscope 108 for access to a luminal network of the patient P. Specifically, catheter 102 of catheter guide assembly 106 may be inserted into a working channel of bronchoscope 108 for navigation through a patient's luminal network. The catheter 102 may itself include imaging capabilities via an integrated camera or optics component 109 and a separate bronchoscope 108 is not strictly required. A locatable guide (LG) 110 (a second catheter), including a sensor 104 may be inserted into catheter 102 and locked into position such that sensor 104 extends a desired distance beyond the distal tip of catheter 102. The position and orientation of sensor 104 relative to a reference coordinate system, and thus the distal portion of catheter 102, within an electromagnetic field can be derived. Catheter guide assemblies 106 are currently marketed and sold by Medtronic PLC under the brand names SUPERDIMENSION® Procedure Kits, ILLUMISITE™ Endobronchial Procedure Kit, ILLUMISITE™ Navigation Catheters, or EDGE™ Procedure Kits, and are contemplated as useable with the disclosure.

System 100 generally includes an operating table 112 configured to support a patient P and monitoring equipment 114 coupled to bronchoscope 108 or catheter 102 (e.g., a video display, for displaying the video images received from the video imaging system of bronchoscope 108 or the catheter 102); a locating or tracking system 115 including a locating module 116, a plurality of reference sensors 18 and a transmitter mat 120 including a plurality of incorporated markers; and a computing device 122 including software and/or hardware used to facilitate identification of a target, pathway planning to the target, navigation of a medical device to the target, and/or confirmation and/or determination of placement of catheter 102, or a suitable device therethrough, relative to the target.

As is typical of catheter guide assembly 10 navigation the six degrees-of-freedom electromagnetic locating or tracking system 115, or other suitable system for determining position and orientation of a distal portion of the catheter 102, is utilized, as will be outlined below for performing registration of a detected position of the sensor 104 and a 3D model generated from a CT or MRI image scan. Tracking system 114 includes the tracking module 116, a plurality of reference sensors 118, and the transmitter mat 120 (including the markers). Tracking system 114 is configured for use with a locatable guide 110 and particularly sensor 104. As described above, locatable guide 110 and sensor 104 are configured for insertion through catheter 102 into patient P′s airways (either with or without bronchoscope 108) and are selectively lockable relative to one another via a locking mechanism.

Transmitter mat 120 is positioned beneath patient P. Transmitter mat 120 generates an electromagnetic field around at least a portion of the patient P within which the position of a plurality of reference sensors 118 and the sensor 104 can be determined with use of a tracking module 116. A second electromagnetic sensor 126 may also be incorporated into the end of the catheter 102. The second electromagnetic sensor 126 may be a five degree-of-freedom sensor or a six degree-of-freedom sensor. One or more of reference sensors 118 are attached to the chest of the patient P. Registration is generally performed to coordinate locations of the three-dimensional model and two-dimensional images from the planning phase, with the patient P′s airways as observed through the bronchoscope 108 and allow for the navigation phase to be undertaken with knowledge of the location of the sensor 104.

Registration of the patient P's location on the transmitter mat 120 may be performed by moving sensor 104 through the airways of the patient P. More specifically, data pertaining to locations of sensor 104, while locatable guide 110 is moving through the airways, is recorded using transmitter mat 120, reference sensors 118, and tracking system 114. A shape resulting from this location data is compared to an interior geometry of passages of a 3D model, and a location correlation between the shape and the 3D model based on the comparison is determined, e.g., utilizing the software on computing device 122. In addition, the software identifies non-tissue space (e.g., air filled cavities) in the three-dimensional model. The software aligns, or registers, an image representing a location of sensor 104 with the three-dimensional model and/or two-dimensional images generated from the three-dimension model, which are based on the recorded location data and an assumption that locatable guide 110 remains located in non-tissue space in patient P's airways. Alternatively, a manual registration technique may be employed by navigating the bronchoscope 108 with the sensor 104 to pre-specified locations in the lungs of the patient P, and manually correlating the images from the bronchoscope to the model data of the three-dimensional model.

Though described herein with respect to EMN systems using EM sensors, the instant disclosure is not so limited and may be used in conjunction with flexible sensors such as fiber-Bragg grating sensors, inertial measurement unit (IMU), ultrasonic sensors, or without sensors. Further, one or more dyes including methylene blue, indocyanine green (ICG), or other fluorescing dyes, may be employed with or without application of near infrared light and optics capable of detecting the fluorescence in order to provide guidance to a desired segment of interest within the patient. Additionally, as outlined below the methods described herein may be used in conjunction with robotic systems such that robotic actuators or manipulators drive the catheter 102 or bronchoscope 108 proximate the target.

In accordance with aspects of the disclosure, the visualization of intra-body navigation of a medical device (e.g., a biopsy tool or a therapy tool), towards a target (e.g., a lesion) may be a portion of a larger workflow of a navigation system. An imaging device 124 (e.g., a CT imaging device such as a cone-beam computed tomography (CBCT) device, including but not limited to Medtronic plc's the O-arm™ system) capable of acquiring 2D and 3D images or video of the patient P is also included in this particular aspect of system 100. The images, sequence of images, or video captured by imaging device 124 may be stored within the imaging device 124 or transmitted to computing device 122 for storage, processing, and display. Additionally, imaging device 124 may move relative to the patient P so that images may be acquired from different angles or perspectives relative to patient P to create a sequence of images, such as a fluoroscopic video. The pose of imaging device 124 relative to patient P while capturing the images may be estimated via markers incorporated with the transmitter mat 120. The markers are positioned under patient P, between patient P and operating table 112 and between patient P and a radiation source or a sensing unit of imaging device 124. The markers incorporated with the transmitter mat 120 may be two separate elements which may be coupled in a fixed manner or alternatively may be manufactured as a single unit. Imaging device 124 may include a single imaging device or more than one imaging device.

Computing device 122 may be any suitable computing device including a processor and storage medium, wherein the processor is capable of executing instructions stored on the storage medium. Computing device 122 may further include a database configured to store patient data, CT data sets including CT images, fluoroscopic data sets including images and video, 3D reconstruction, navigation plans, and any other such data. Although not explicitly illustrated, computing device 122 may include inputs, or may otherwise be configured to receive, CT data sets, fluoroscopic images/video and other data described herein. Additionally, computing device 122 includes a display configured to display graphical user interfaces. Computing device 122 may be connected to one or more networks through which one or more databases may be accessed.

Though described herein in connection with bronchoscopic and catheter-based navigation systems that are often handheld and user actuated, the disclosure is not so limited. The systems and methods described herein may be motor driven via user interaction or robotically driven whether via user interaction of a controller or by autonomous or semi-autonomous robotic systems without departing from the scope of the disclosure.

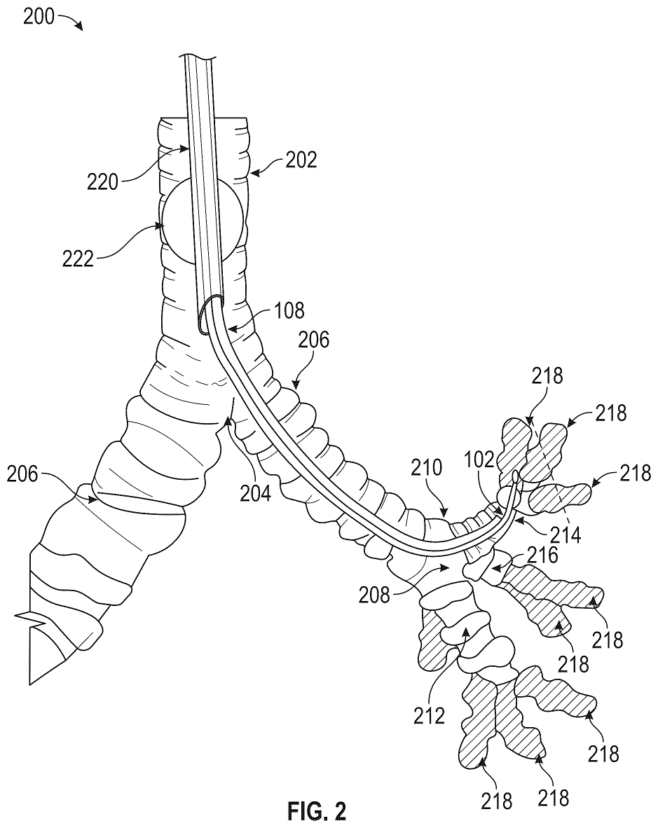

FIG. 2 depicts a schematic of a patient's airways 200. In particular FIG. 2 depicts the features of the left lung. The airways 200 starts at the trachea 202, which bifurcates at the main carina 204 from which the left main bronchus and right main bronchus 206 descend. Focusing on the left main bronchus 206, at the next bifurcation 208, the superior lobar bronchus 210 separates from the inferior lobar bronchus 212. Though not clearly shown in FIG. 2, the superior lobar bronchus 210 bifurcates into the superior division bronchus 214 and the lingular division bronchus 216. From the superior division bronchus 214 and lingular division bronchus branch the segmental bronchi 218 that supply air to the lung segments of the upper lobe of the left lung. Similarly, branching from the inferior lobar bronchus 214 are segmental bronchus 218 that supply air to the segments of the left lower lobe. As is known, the right lobe has similar physiology but with the bronchi separating into three lung lobes.

During a bronchoscopic procedure an intubation tube 220 is inserted within the trachea 202 and a balloon 222 thereon is inflated to hold the intubation tube stably in the trachea 202. As shown in FIG. 2 a bronchoscope 108 is inserted into the intubation tube 220. Employing either a pathway plan developed using pre-procedure imaging and navigation system, as described above, intra-procedural imaging such as fluoroscopy, or the imaging capabilities of the bronchoscope 108, the bronchoscope is navigated until it becomes wedged in the airways. In FIG. 2 the bronchoscope 108 became wedged in superior division bronchus 214. A catheter 102 is inserted through the bronchoscope 108 via a working channel. The catheter 102 is extended from the bronchoscope 108 and advanced to the desired segmental bronchus 218.

Those of skill in the art will understand that a bronchoscope 108 is not required and that the catheter 102 may be navigated to the desired segmental bronchus 218 without the need of a separate bronchoscope 108, particularly when equipped with a camera 109, as described above.

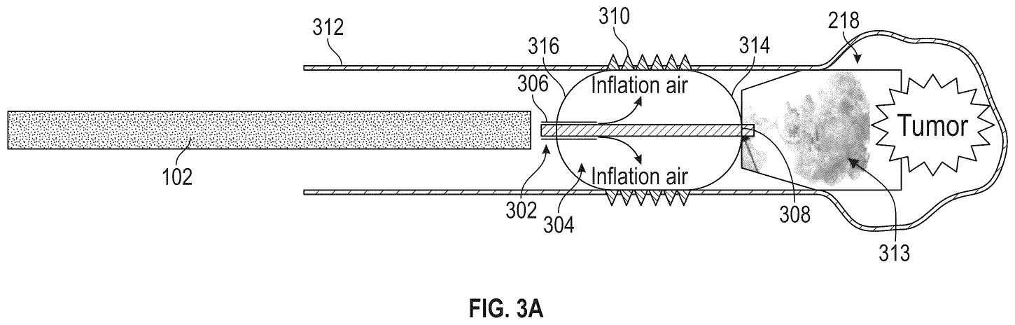

FIG. 3A depicts a distal portion of the catheter 102. The catheter 102 is a portion of a therapy application system including a therapeutic medium source, a vacuum source, and an inflation source, all retained outside of the patient and in fluid communication with the catheter 102 as described below. As noted above, the catheter 102 includes a working channel therein (not shown) through which the LG 110 may be inserted if employed. If the LG 110 is employed, it may be removed and a dual lumen catheter 302 inserted therein. Alternatively, the catheter 102 may be navigated to or near the desired segmental bronchus 218 with the dual lumen catheter 302 located therein. The dual lumen catheter 302 is advanced from the catheter 102 to a desired location (e.g., just past a bifurcation and into a desired segmental bronchus 218). Once the dual lumen catheter 302 is so positioned, a balloon 304 is inflated. The dual lumen catheter 102 is formed in this example from an outer tube 306 and an inner tube 308. The space between the inner tube 308 and outer tube 306 is the first lumen and the second lumen is formed inside of the inner tube 308. The outer tube 306 may be connected to an inflation source containing inflation medium (e.g., gas or liquid). Forcing of the inflation medium through the outer lumen (between the outer tube 306 and inner tube 308) and into the balloon 304 inflates the balloon 304 and forces securing features 310 (barbs, hooks, micro-barbs, or others means) formed on the exterior of the balloon 304 to engage with the airway 312 and secure the balloon 304 in the airway 312 at the desired location.

Though depicted as two coaxial tubes (outer tube 306 and inner tube 308), the disclosure is not so limited and other configurations are contemplated within the disclosure. For example, the coaxial tubes instead be configured as two equally sized adjacent tubes without departing from the scope of the disclosure. Those of skill in the art will also recognize that inner tube 308 may be a needle or other therapy injection device capable of traversing the balloon 304 and the valves 402 without departing from the scope of the disclosure.

In accordance with aspects of the disclosure, rather than or in addition to a mechanical structure such as a barb or spike, an exterior surface of the balloon 304 is coated with an adhesive material which bonds to the epithelium of the airways. This bonding, in combination with the force applied by the inflation medium holds the balloon 304 in place following deployment. Still further, an expanding metal structure, similar to a stent, may be employed to secure the exterior of the balloon 304 within the airways.

As shown in FIG. 3A, inner tube 308 extends through the balloon 304 and exits on a treatment side of the balloon 304. The inner tube 308 is connected and in fluid communication with a therapeutic medium source located outside of the patient. After inflation of the balloon 304 and the securing features 310 engaging the airways 312, placement of the balloon 304 within the airways can be optionally confirmed with imaging such as fluoroscopic imaging, computed tomography, or cone beam computed tomography or other imaging modalities. With the positioning confirmed, the therapeutic medium 313 can be forced from the therapeutic medium source through the inner tube 308 and into the segmental bronchus 218 that is distal of the balloon 304. The balloon 304 prevents the therapeutic medium from escaping the desired segmental bronchus 218 and effectively isolates that segmental bronchus 218 from the rest of the airways and lung structures.

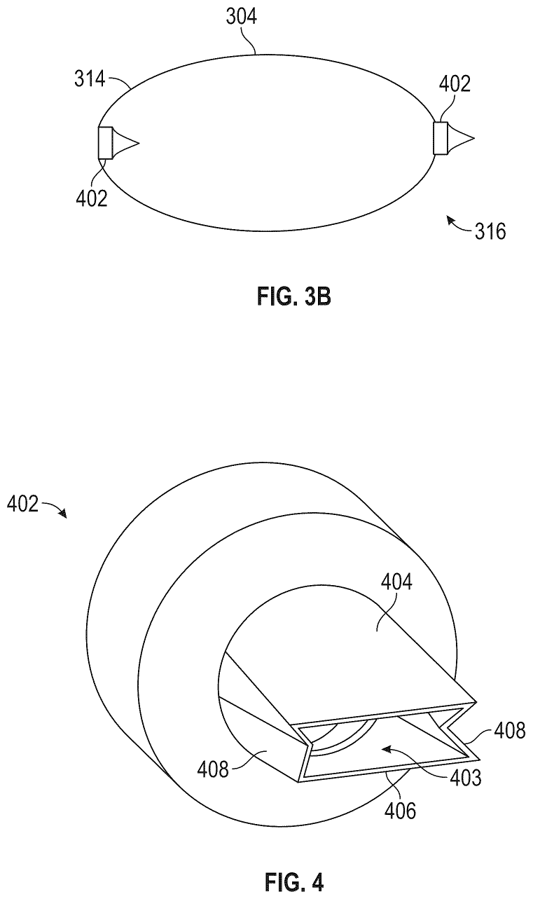

Once the therapeutic medium is injected into the segmental bronchus 218, the dual lumen catheter 302 can be withdrawn from the balloon 304, as shown in FIG. 3B. The balloon 304 may include a valve 402 (e.g., a duck bill valve or other self-sealing valve, see FIG. 4) on both the proximal end 314 and the distal end 316 of the balloon 304. The valves 402 allows for the dual lumen catheter to be withdrawn from the balloon 304 without allowing the therapeutic medium to enter the balloon 304 or allow the balloon 304 to deflate. The dual lumen catheter 302 is removed and the patient may return to their normal activities, though some patients may have some restrictions (e.g., no airplane travel, contact sports, heavy lifting, etc.). As shown in FIG. 4 the valve 402 includes an opening 403, a top bill 404, a bottom bill 406, and two pleated sides 408. The pleated 408 sides allow the top bill 404 and the bottom bill 406 to separate, for example, when the inner tube 308 is passed therebetween. Upon removal of the inner tube 308, the pleated sides 408 are configured to collapse and prevent both the escape of inflation medium from the balloon 304 into the segmental bronchus 218, and the escape of therapeutic medium from the segmental bronchus into the balloon 304. Valve 402 is just one example of a valve that may be used for these purposes, others include for example sports balls valves that allow the passage of a needle similar to the inner tube 308 for the inflation of a sports ball, but seal upon removal of the needle to prevent deflation.

In one aspect of the disclosure, during the initial navigation and placement of the balloon 304, the balloon 304 is held in place on the dual lumen catheter 302 by a combination of friction forces applied by one or more valves 402 and application of a vacuum to a proximal portion of the outer tube 306. This vacuum causes the balloon 304 to be held in a collapsed state on the inner tube 308. As will be appreciated, the inner tube 308 may initially be kept within the balloon 304 to maintain the vacuum during navigation and placement, and then advanced through the valve 402 on the distal end 316 of the balloon for release of the therapeutic medium 313.

Because the balloon 304 stays inflated, the therapeutic medium 313 is isolated in the segmental bronchus 218 receiving treatment and prevents the therapeutic medium from leaking into other portions of the airways. The therapeutic medium remains in the desired segmental bronchus 218 for a desired period of time (e.g., 2-4 weeks). In some instances, the therapeutic medium is retained within the segmental bronchus for at least beyond the average cellular doubling rate of between about 17-25 days.

Though described as employing an inner tube 308 that is coaxial with outer tube 306, the disclosure is not so limited. As an alternative, the inner tube 308 may be a component of the balloon 304. The rigid component incorporates the distal valve 402 and having a channel for the air travelling through proximal valve 402. In this manner, the interior passage of 308 is never in contact with the interior of the balloon 304.

Still further, the catheter 102 may include a mechanical capturing feature. The mechanical capturing feature secures the balloon 304 to the catheter 102. Thus, the balloon may be inflated, the therapeutic medium 313 deployed and the inner tube 308 and outer tube 306 retracted within the catheter 102 without release of the balloon 304. In this manner, placement and security of the balloon 304 may be confirmed prior to disengagement of the balloon 304 from the catheter 102.

As described above, the progression of the therapy may be observed via imaging such as CT, MRI, fluoroscopy, and others to assess the effects the therapeutic medium has on the tumor or lesion located within the isolated portion of the airways. If further therapy is required, a dual lumen catheter 302 may again be advanced into the airways of the patient, through valves 402 on the balloon 304 to again achieve the orientation depicted in FIG. 3A. Additional therapeutic medium may be injected via the inner tube 308, and additional inflation medium may also be injected via the outer tube 306 to ensure the balloon is fully inflated.

If, however, the therapy is complete, vacuum is applied to the inner tube 308 to suction the therapeutic medium and any secretions or tissues that have released from the isolated segmental bronchus 218 as a result of the treatment. Next, a vacuum is applied to the outer tube 306 to deflate the balloon 304. By application of sufficient vacuum and holding the vacuum constant, the balloon 304 is drawn onto the inner tube 308, and the dual lumen catheter 302 and balloon 304 can be safely removed from the patient. In some instances, a grasping tool (not shown) may be deployed from the bronchoscope 108 to retrieve the balloon 304, for example under fluoroscopic image guidance, if needed. Once removed, the previously isolated segmental bronchus 218 begins receiving air and commences returning to their normal functions.

The therapeutic medium may include one or more marker materials. The marker materials may be for example visible under imaging including fluoroscopy, magnetic resonance imaging (MRI), computed tomography (CT), cone beam computed tomography (CBCT), positron emissions tomography (PET) and others. Following isolation of the therapeutic medium in the desired segmental bronchus 218, the patient may be periodically imaged using one of these techniques to identify the incidence of any systemic drainage (e.g., via the lymph nodes, or via capillary action). This assessment can lead to the determination that application of additional therapeutic medium is appropriate, as described above.

Additionally or alternatively, the therapeutic agent may include one or more biologic markers specified to bind with the cells forming the tumor or lesion. For example, the biologic marker may bind with specific proteins found only in the cancerous cells and not healthy tissue. Again, through one or more of the imaging techniques described above, the progress of the therapy may be assessed, and additional therapeutic medium can be added, or cessation of the therapy determined based on the imaging of the patient and the identification of the location, concentration, pattern of the biological markers. For example, the images may be analyzed to determine the distribution of the biological marker if there is a concentration in one location, that is indicative of the location of the tumor or lesion. However, if the biological markers are relatively evenly distributed throughout the isolated segmental bronchus, then the tumor or lesion may be determined to be fully treated.

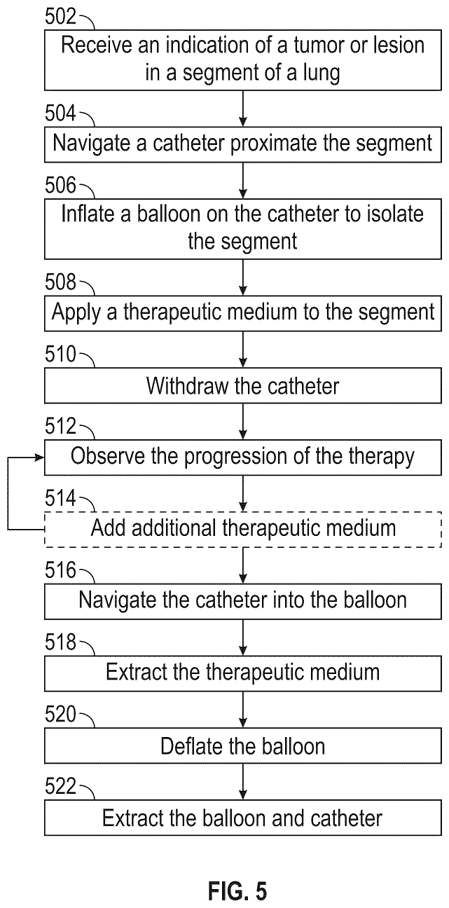

FIG. 5 depicts a method in accordance with the disclosure. At step 502 an indication is received by a clinician that a patient has a tumor or lesion that is accessible via a lung segment and is amenable for an isolated segment therapy. At step 504 a catheter 102 is navigated proximate a segment in which the tumor or lesion resides. The catheter 102 may be navigated, for example, to a location just beyond the bifurcation of the airways separating the segment in which the tumor or lesion resides from other segments of the lungs. At step 506 a balloon 304 on the catheter 102 is inflated isolating the segment in which the tumor or lesion resides from the remainder of the airways. Following isolation, a therapeutic medium is released for a therapeutic medium source and floods into the segment in which the tumor or lesion resides at step 508. The balloon 304, isolating the segment in which the tumor or lesion resides, prevents the therapeutic medium from escaping to other portions of the airways. The catheter 102 is withdrawn at step 510 with the balloon 304 left in place isolating the segment of the lungs in which the tumor or lesion resides. At step 512, the therapy may be periodically observed using one or more imaging modalities, as described herein above. Optionally, at step 514 the catheter 102 may be navigated through the airways, and additional therapeutic medium may be applied to the segment in which the tumor or lesion resides. At the conclusion of the therapy, for example as assessed at step 512, the catheter 102 may again be navigated into the balloon 304 at step 516. The catheter 102 is employed to remove any remaining therapeutic media, other secretions, and any tissue or matter from the segment in which the tumor or lesion once resided at step 518. Next, the catheter 102 is employed to deflate the balloon 304 at step 520 and the balloon and catheter are extracted at step 522.

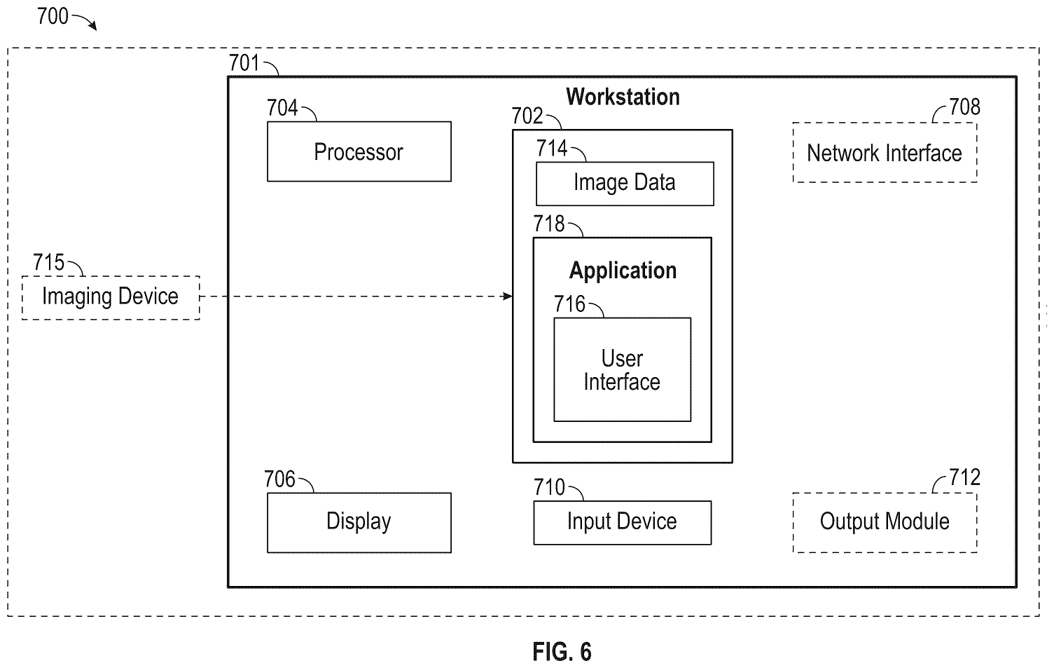

Reference is now made to FIG. 6, which is a schematic diagram of a system 700 configured for use with the methods of the disclosure including the method of FIG. 4. System 700 may include a workstation 701, and optionally an imaging device 715 (e.g., a fluoroscope or an ultrasound device). In some embodiments, workstation 701 may be coupled with imaging device 715, directly or indirectly, e.g., by wireless communication. Workstation 701 may include a memory 702, a processor 704, a display 706 and an input device 710. Processor or hardware processor 704 may include one or more hardware processors. Workstation 701 may optionally include an output module 712 and a network interface 708. Memory 702 may store an application 718 and image data 77. Application 718 may include instructions executable by processor 704 for executing the methods of the disclosure including the method of FIG. 4.

Application 718 may further include a user interface 716. Image data 714 may include the CT scans, the generated fluoroscopic 3D reconstructions of the target area and/or any other fluoroscopic image data and/or the generated one or more slices of the 3D reconstruction. Processor 704 may be coupled with memory 702, display 706, input device 710, output module 712, network interface 708 and imaging device 715. Workstation 701 may be a stationary computing device, such as a personal computer, or a portable computing device such as a tablet computer. Workstation 701 may embed a plurality of computer devices.

Memory 702 may include any non-transitory computer-readable storage media for storing data and/or software including instructions that are executable by processor 704 and which control the operation of workstation 701 and, in some embodiments, may also control the operation of imaging device 715. Imaging device 715 may be used to capture a sequence of fluoroscopic images based on which the fluoroscopic 3D reconstruction is generated and to capture a live 2D fluoroscopic view according to this disclosure. In an embodiment, memory 702 may include one or more storage devices such as solid-state storage devices, e.g., flash memory chips. Alternatively, or in addition to the one or more solid-state storage devices, memory 702 may include one or more mass storage devices connected to the processor 704 through a mass storage controller (not shown) and a communications bus (not shown).

Although the description of computer-readable media contained herein refers to solid-state storage, it should be appreciated by those skilled in the art that computer-readable storage media can be any available media that can be accessed by the processor 704. That is, computer readable storage media may include non-transitory, volatile, and non-volatile, removable, and non-removable media implemented in any method or technology for storage of information such as computer-readable instructions, data structures, program modules or other data. For example, computer-readable storage media may include RAM, ROM, EPROM, EEPROM, flash memory or other solid-state memory technology, CD-ROM, DVD, Blu-Ray or other optical storage, magnetic cassettes, magnetic tape, magnetic disk storage or other magnetic storage devices, or any other medium which may be used to store the desired information, and which may be accessed by workstation 1001.

Application 718 may, when executed by processor 704, cause display 706 to present user interface 716. User interface 716 may be configured to present to the user a single screen including a three-dimensional (3D) view of a 3D model of a target from the perspective of a tip of a medical device, a live two-dimensional (2D) fluoroscopic view showing the medical device, and a target mark, which corresponds to the 3D model of the target, overlaid on the live 2D fluoroscopic view. User interface 716 may be further configured to display the target mark in different colors depending on whether the medical device tip is aligned with the target in three dimensions.

Network interface 708 may be configured to connect to a network such as a local area network (LAN) consisting of a wired network and/or a wireless network, a wide area network (WAN), a wireless mobile network, a Bluetooth network, and/or the Internet. Network interface 708 may be used to connect between workstation 701 and imaging device 715. Network interface 708 may also be used to receive image data 714. Input device 710 may be any device by which a user may interact with workstation 701, such as, for example, a mouse, keyboard, foot pedal, touch screen, and/or voice interface. Output module 712 may include any connectivity port or bus, such as, for example, parallel ports, serial ports, universal serial busses (USB), or any other similar connectivity port known to those skilled in the art. From the foregoing and with reference to the various figures, those skilled in the art will appreciate that certain modifications can be made to the disclosure without departing from the scope of the disclosure.

### EXAMPLES

The disclosure may be further described in connection with the following examples:

Example 1—A therapy application system comprising:
a dual lumen catheter including a first tube and a second tube, the dual lumen catheter configured for navigation within airways of a patient;
a balloon formed on a distal portion of the dual lumen catheter, the balloon including a first valve on a proximal portion of the balloon and a second valve on a distal portion of the balloon;
an inflation source in fluid communication with the first tube; and
a therapeutic medium source in fluid communication with second tube, wherein the dual lumen catheter extends past the first valve and through the balloon, the first tube being in fluid communication with the balloon and release of inflation medium from the inflation source expands the balloon and isolates a segmental bronchus, and wherein the second tube extends beyond the second valve and release of the therapeutic medium from the therapeutic medium source floods the isolated segmental bronchus.

Example 2—The therapy application system of example 1, further comprising a vacuum source in fluid communication with the second tube and configured to extract the therapeutic medium from the isolated segmental bronchus.

Example 3—The therapy application system of one of the preceding examples, wherein the vacuum source is in fluid communication with the first tube to deflate the balloon.

Example 4—The therapy application system of example 3, wherein the vacuum applied to the balloon secures the balloon to the dual lumen catheter for removal from the airways of the patient.

Example 5—The therapy application system of one of the preceding claims, further comprising securing features on an exterior surface of the balloon, wherein the securing features is one or more of an adhesive, micro-barbs, hooks, or spikes.

Example 6—The therapy application system of one of the preceding claims, wherein the catheter includes an electromagnetic (EM) sensor.

Example 7—The therapy application system of example 6, further comprising an EM transmitting matt, wherein the sensor on the catheter detects an EM field generated by the EM transmitting matt.

Example 8—The therapy application system of example 7, further comprising an application stored in a memory and executed by a processor, wherein the application receives a signal representative of the detected EM field from the EM sensor and presents on a user interface a representation of a location of the catheter within the airways of the patient.

Example 9—The therapy application system of example 8, wherein the memory stores therein images of the airways of the patient and displays the location of the catheter in a three-dimensional model generated from the stored images.

Example 10—The therapy application system of example 9, further comprising an intraprocedural imaging device.

Example 11—The therapy application system of example 10, wherein the intraprocedural imaging device an optical sensor on a distal portion of the catheter.

Example 12—The therapy application system of example 10, wherein the intraprocedural imaging device is a fluoroscope, computed tomography device, a cone beam computed tomography device, a magnetic resonance imaging device, or a positron emissions tomography device.

Example 13—The therapy application system of example 10, wherein captured intraprocedural images are analyzed by the application to assess the progression of the therapy.

Example 14—The therapy application system of example 10, wherein captured intraprocedural images are analyzed by the application to assess the placement the catheter. Example 15—The therapy application system of one of the preceding claims, further comprising a robotic manipulator operably connected to the catheter for placement of the catheter within the airways of the patient. While detailed embodiments are disclosed herein, the disclosed embodiments are merely examples of the disclosure, which may be embodied in various forms and aspects. For example, embodiments of an electromagnetic navigation system. Therefore, specific structural and functional details disclosed herein are not to be interpreted as limiting, but merely as a basis for the claims and as a representative basis for teaching one skilled in the art to variously employ the disclosure in virtually any appropriately detailed structure.

We claim:

1. A method of applying therapy comprising:
navigating a dual lumen catheter to a desired segmental bronchus;
inflating a balloon located on a distal portion of the catheter via a first tube of the dual lumen catheter to isolate the segmental bronchus;
injecting a therapeutic medium into the isolated segmental bronchus via a second tube of the dual lumen catheter; and
retracting the dual lumen catheter to leave the balloon indwelling, wherein the balloon retains the injected therapeutic medium in the isolated segmental bronchus.

2. The method of claim 1, wherein inflation of the balloon engages securing features with the segmental bronchus to hold the balloon in place.

3. The method of claim 2, wherein the securing features are micro-barbs.

4. The method of claim 2, wherein the securing features are an adhesive coating formed on an exterior surface of the balloon that adheres to an airway wall of the segmental bronchus.

5. The method of claim 1, further comprising imaging the segmental bronchus with the balloon indwelling to assess the progression of the therapy.

6. The method of claim 5, further comprising determining whether the therapeutic medium is draining from the segmental bronchus.

7. The method of claim 5, further comprising analyzing a distribution of a biological marker, wherein the biological marker is configured to bind with a tumor or lesion.

8. The method of claim 1, further comprising navigating the dual lumen catheter to the indwelling balloon.

9. The method of claim 8, further comprising passing the dual lumen catheter through a first valve on the balloon, wherein the first tube is configured to supply additional inflation medium into the balloon.

10. The method of claim 8, further comprising passing the second tube through a second valve on the balloon.

11. The method of claim 10, further comprising injecting additional therapeutic medium into the isolated segmental bronchus.

12. The method of claim 10, further comprising extracting the therapeutic medium from the segmental bronchus.

13. The method of claim 10, further comprising deflating the balloon.

14. The method of claim 13, wherein the deflation comprises applying a vacuum to the balloon, wherein the vacuum secures the balloon to a portion of the dual lumen catheter.

15. The method of claim 13, further comprising extracting the dual lumen catheter and balloon from the segmental bronchus.

16. A therapy application system comprising:
a dual lumen catheter including a first tube and a second tube, the dual lumen catheter configured for navigation within airways of a patient;
a balloon formed on a distal portion of the dual lumen catheter, the balloon including a first valve on a proximal portion of the balloon and a second valve on a distal portion of the balloon;
an inflation source in fluid communication with the first tube; and
a therapeutic medium source in fluid communication with second tube, wherein the dual lumen catheter extends past the first valve and into the balloon, the first tube being in fluid communication with the balloon and release of inflation medium from the inflation source expands the balloon and isolates a segmental bronchus, and wherein the second tube extends beyond the second valve and release of the therapeutic medium from the therapeutic medium source floods the isolated segmental bronchus.

17. The therapy application system of claim 16, further comprising a vacuum source in fluid communication with the second tube and configured to extract the therapeutic medium from the isolated segmental bronchus.

18. The therapy application system of claim 17, wherein the vacuum source is in fluid communication with the first tube to deflate the balloon.

19. The therapy application system of claim 18, wherein the vacuum applied to the balloon secures the balloon to the dual lumen catheter for removal from the airways of the patient.

20. The therapy application system of claim 16, further comprising securing features on an exterior surface of the balloon, wherein the securing features is one or more of an adhesive, micro-barbs, hooks, or spikes.
